(19) United States
(12) Patent Application Publication
(10) Pub. No.: US 2014/0330373 A1
(43) Pub. Date: Nov. 6, 2014

(54) Reinforced Prosthetic Tissue Valves

(71) Applicants: Robert G. Matheny, Norcross, GA (US); Anna Fallon, Woodstock, GA (US)

(72) Inventors: Robert G. Matheny, Norcross, GA (US); Anna Fallon, Woodstock, GA (US)

(21) Appl. No.: 14/264,308

(22) Filed: Apr. 29, 2014

Related U.S. Application Data
(60) Provisional application No. 61/819,250, filed on May 3, 2013.

(51) Int. Cl. A61F 2/24; A61L 27/36; A61L 27/54

(52) U.S. Cl. CPC A61F 2/2418; A61L 27/54; A61L 27/3604
USPC 623/2.36

(57) ABSTRACT

A prosthetic atrioventricular valve that includes a continuous tubular member having proximal and distal ends, the tubular member comprising an extracellular matrix (ECM) material, and a reinforcement member disposed in the tubular member lumen and attached to the distal end of the tubular member, the reinforcement member comprising at least two elongated linear members that are configured to connect the joined tubular and reinforcement members to a cardiovascular structure.

### CROSS-REFERENCES TO RELATED APPLICATIONS

This application claims the benefit of U.S. Application No. 61/819,250, filed on May 3, 2013.

### FIELD OF THE INVENTION

The present invention generally relates to prosthetic valves for replacing defective cardiovascular valves. More particularly, the present invention relates to reinforced prosthetic atrioventricular valves and methods for anchoring same to cardiovascular structures and/or tissue.

### BACKGROUND OF THE INVENTION

As is well known in the art, the human heart has four valves that control blood flow circulating through the human body. Referring to FIGS. 1A and 1B, on the left side of the heart 100 is the mitral valve 102, located between the left atrium 104 and the left ventricle 106, and the aortic valve 108, located between the left ventricle 106 and the aorta 110. Both of these valves direct oxygenated blood from the lungs into the aorta 110 for distribution through the body.

The tricuspid valve 112, located between the right atrium 114 and the right ventricle 116, and the pulmonary valve 118, located between the right ventricle 116 and the pulmonary artery 120, however, are situated on the right side of the heart 100 and direct deoxygenated blood from the body to the lungs.

There are also five papillary muscles in the heart 100; three in the right ventricle 116 and two in the left ventricle 106. The anterior, posterior and septal papillary muscles of the right ventricle 116 each attach via chordae tendinae to the tricuspid valve 112. The anterior and posterior papillary muscles of the left ventricle 106 attach via chordae tendinae to the mitral valve 102.

Since heart valves are passive structures that simply open and close in response to differential pressures, the issues that can develop with valves are typically classified into two categories: (i) stenosis, in which a valve does not open properly, and (ii) insufficiency (also called regurgitation), in which a valve does not close properly. Stenosis and insufficiency can occur concomitantly in the same valve or in different valves.

Both of the noted valve abnormalities can adversely affect organ function and result in heart failure. For example, insufficiency of the inlet (atrioventricular) tricuspid valve 112 to the right ventricle 116 of the heart 100 results in regurgitation of blood back into the right atrium 114, which, serving to receive blood flow returning in the veins from the entire body, then results in turn in suffusion and swelling (edema) of all the organs, most notably in the abdomen and extremities, insufficient forward conduction of blood flow from the right ventricle 116 into the lungs causing compromise of pulmonary function, and ultimately pump failure of the right heart. Collectively these conditions (collectively deemed right heart failure) can, and in many instances will. lead to incapacity and. possibly, death if progressive and uncorrected.

In addition to stenosis and insufficiency of a heart valve, surgical intervention may also be required for certain types of bacterial or fungal infections, wherein the valve may continue to function normally, but nevertheless harbors an overgrowth of bacteria (i.e. “vegetation”) on the valve leaflets. The vegetation can, and in many instances will, flake off (i.e. “embolize”) and lodge downstream in a vital artery.

If such vegetation is present on the valves of the left side (i.e., the systemic circulation side) of the heart, embolization can, and often will, result in sudden loss of the blood supply to the affected body organ and immediate malfunction of that organ. The organ most commonly affected by such embolization is the brain, in which case the patient can, and in many instances will, suffer a stroke.

Likewise, bacterial or fungal vegetation on the tricuspid valve can embolize to the lungs. The noted embolization can, and in many instances will, result in lung dysfunction.

Treatment of the noted heart valve dysfunctions typically comprises reparation of the diseased heart valve with preservation of the patient's own valve or replacement of the valve with a mechanical or bioprosthetic valve, i.e. a prosthetic valve.

Various prosthetic heart valves have thus been developed for replacement of natural diseased or defective heart valves. Illustrative are the tubular prosthetic tissue valves disclosed in Applicant's Co-Pending U.S. application Ser. Nos. 13/560,573, 13/782,024, 13/782,289, 13/804,683, 13/182,170, 13/480,347 and 13/480,324. A further tubular prosthetic valve is disclosed in U.S. Pat. Nos. 8,257,434 and 7,998,196.

Heart valve replacement requires a great deal of skill and concentration to achieve a secure and reliable attachment of a prosthetic valve to a cardiovascular structure or tissue. Various surgical methods for implanting a prosthetic valve have thus been developed.

The most common surgical method that is employed to implant a prosthetic atrioventricular valve (mitral or tricuspid) comprises suturing a segment of one or more leaflets directly to the anterior and/or posterior papillary muscles.

A major problem associated with such attachment is that the papillary muscles and the region proximate thereto are subject to extreme stress (induced by cardiac cycles), which can, and in most instances will, adversely affect the structural integrity of the valve.

There is thus a need to provide improved prosthetic atrioventricular tissue valves and methods for attaching same to cardiovascular structures and/or tissue that maintain or enhance the structural integrity of the valve when subjected to cardiac cycle induced stress.

It is therefore an object of the present invention to provide “reinforced” prosthetic atrioventricular valves and methods for implanting same that overcome the drawbacks and disadvantages associated with conventional prosthetic atrioventricular valves.

It is another object of the present invention to provide reinforced prosthetic atrioventricular tissue valves and methods for attaching same to cardiovascular structures and/or tissue that enhance the structural integrity of the valve.

It is another object of the present invention to provide reinforced prosthetic atrioventricular tissue valves that exhibit optimum mechanical compatibility with cardiovascular structures.

It is another object of the present invention to provide reinforced prosthetic atrioventricular tissue valves and methods for attaching same to cardiovascular structures and/or tissue that preserve the structural integrity of the cardiovascular structure(s) when attached thereto.

It is another object of the present invention to provide reinforced prosthetic atrioventricular tissue valves having means for secure, reliable, and consistently highly effective attachment to cardiovascular structures and/or tissue.

It is another object of the present invention to provide improved methods for securely attaching prosthetic atrioventricular valves to cardiovascular structures and/or tissue.

It is another object of the present invention to provide reinforced prosthetic atrioventricular tissue valves that induce host tissue proliferation, bioremodeling and regeneration of new tissue and tissue structures with site-specific structural and functional properties.

It is another object of the present invention to provide reinforced prosthetic atrioventricular tissue valves that are capable of administering a pharmacological agent to host tissue and, thereby produce a desired biological and/or therapeutic effect.

### SUMMARY OF THE INVENTION

The present invention is directed to prosthetic atrioventricular tissue valves that can be readily employed to selectively replace diseased or defective mitral and tricuspid valves, and methods for attaching (or anchoring) same to cardiovascular structures and/or tissue.

In a preferred embodiment of the invention, the prosthetic atrioventricular valves comprise continuous tubular members having proximal and distal ends.

In a preferred embodiment, the tubular members include a reinforcement member (i.e. tubular member constructs) that is designed and configured to enhance the structural integrity of the tubular member and, hence, prosthetic atrioventricular tissue valve formed therefrom, and facilitate secure engagement of the prosthetic valve to cardiovascular structures, e.g., selective papillary muscles, ventricles, etc., and/or cardiovascular tissue.

In some embodiments, the reinforcement member includes anchoring means.

In a preferred embodiment of the invention, the prosthetic atrioventricular valves (or tubular member constructs) are capable of transitioning from a pre-deployment configuration, wherein the prosthetic valves are capable of being positioned within a cardiovascular vessel, to a post-deployment configuration, wherein the prosthetic valves are disposed proximate host tissue of the vessel.

In some embodiments, the reinforcement member comprises at least one linear member.

In some embodiments, the reinforcement member comprises a multiple-link member.

In some embodiments, the multiple-link member comprises an integral member.

In a preferred embodiment of the invention, the reinforcement member comprises a biocompatible material. In some embodiments, the reinforcement member comprises a biocompatible and biodegradable material.

In some embodiments, the reinforcement member comprises magnesium.

In some embodiments, the reinforcement member comprises stainless steel.

In some embodiments, the reinforcement member comprises a cobalt-chrome nickel alloy.

In some embodiments, the reinforcement member comprises Nitinol®.

In some embodiments of the invention, the reinforcement member comprises an extracellular matrix (ECM) material.

In some embodiments of the invention, the reinforcement member comprises a polymeric material.

In some embodiments of the invention, each link of the multiple-link reinforcement member is formed from the same material.

In some embodiments, each link of the multiple-link reinforcement member is formed from different materials.

In some embodiments of the invention, at least a portion of the reinforcement member is encased in an ECM material.

In some embodiments of the invention, the tubular members comprise an ECM material.

In a preferred embodiment of the invention, the ECM material comprises mammalian extracellular matrix tissue selected from the group comprising small intestine submucosa (SIS), urinary bladder submucosa (UBS), stomach submucosa (SS), central nervous system tissue, epithelium of mesodermal origin, i.e. mesothelial tissue, dermal extracellular matrix, subcutaneous extracellular matrix, gastrointestinal extracellular matrix, i.e. large and small intestines, tissue surrounding growing bone, placental extracellular matrix, ornomentum extracellular matrix, cardiac extracellular matrix, e.g., pericardium and/or myocardium, kidney extracellular matrix, pancreas extracellular matrix, lung extracellular matrix, and combinations thereof.

In some embodiments of the invention, the tubular members (or a portion thereof) and/or reinforcement member encasement material(s) include at least one additional biologically active agent or composition, i.e. an agent that induces or modulates a physiological or biological process, or cellular activity, e.g., induces proliferation, and/or growth and/or regeneration of tissue.

In some embodiments of the invention, the biologically active agent comprises a growth factor.

In some embodiments of the invention, the biologically active agent comprises a cell.

In some embodiments, the tubular members (or a portion thereof) and/or reinforcement member encasement material(s) include at least one pharmacological agent or composition (or drug), i.e. an agent or composition that is capable of producing a desired biological effect in vivo, e.g., stimulation or suppression of apoptosis, stimulation or suppression of an immune response, etc.

In some embodiments of the invention, the pharmacological agent comprises an anti-inflammatory agent.

In some embodiments of the invention, the pharmacological agent comprises a statin, i.e. a HMG-CoA reductase inhibitor.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIGS. 1A and 1B are schematic illustrations of a human heart, showing blood flow therethrough.

FIG. 2 is a perspective view of one embodiment of a reinforced prosthetic atrioventricular tissue valve, in accordance with the invention;

FIG. 3 is a perspective view of another embodiment of a prosthetic atrioventricular tissue valve, in accordance with the invention;

FIG. 4 is a perspective view of a prosthetic valve reinforcement member, in accordance with the invention;

FIG. 5 is a side plane view of the prosthetic valve reinforcement member shown in FIG. 4, in accordance with the invention;

FIG. 6 is a perspective view of one embodiment of a tubular member construct, having a tubular valve member and the reinforcement member shown in FIG. 4, in accordance with the invention;

FIG. 7 is a side plane, sectional view of the tubular member construct shown in FIG. 6;

FIG. 8 is a perspective view of another embodiment of a tubular member construct, having a tubular valve member and the reinforcement member shown in FIG. 4, in accordance with the invention; and

FIG. 9 is a side plane, sectional view of the tubular member construct shown in FIG. 8.

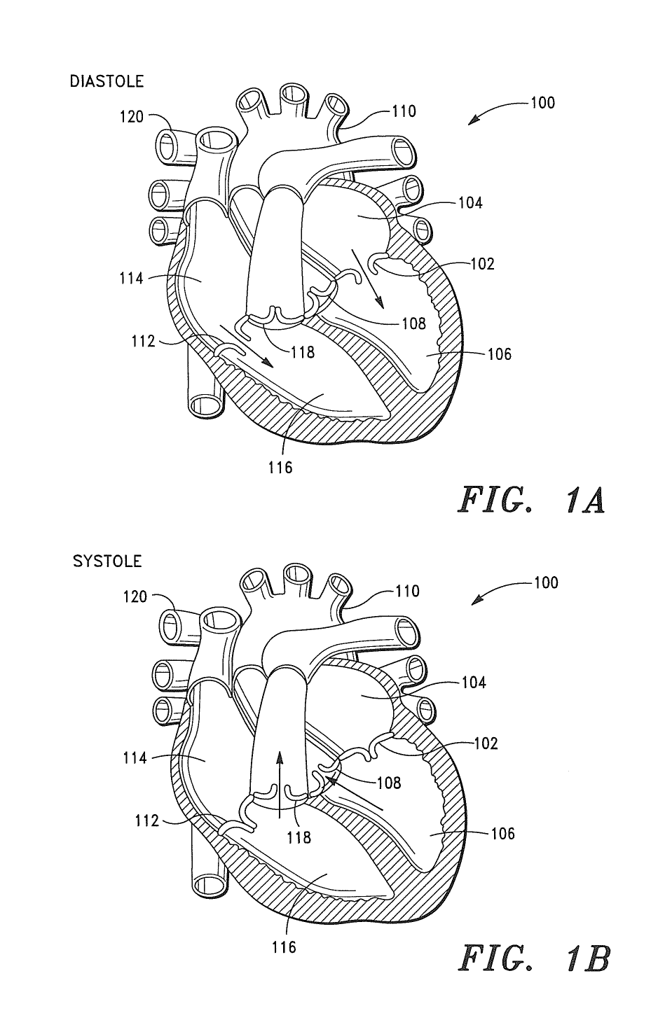

### DETAILED DESCRIPTION OF THE PREFERRED EMBODIMENT

Before describing the present invention in detail, it is to be understood that this invention is not limited to particularly exemplified apparatus, systems, structures or methods as such may, of course, vary. Thus, although a number of apparatus, systems and methods similar or equivalent to those described herein can be used in the practice of the present invention, the preferred apparatus, systems, structures and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

As used in this specification and the appended claims, the singular forms “a, “an” and “the” include plural referents unless the content clearly dictates otherwise. Thus, for example, reference to “a pharmacological agent” includes two or more such agents and the like.

Further, ranges can be expressed herein as from “about” or “approximately” one particular value, and/or to “about” or “approximately” another particular value. When such a range is expressed, another embodiment includes from the one particular value and/or to the other particular value. Similarly, when values are expressed as approximations, by use of the antecedent “about” or “approximately”, it will be understood that the particular value forms another embodiment. It will be further understood that the endpoints of each of the ranges are significant both in relation to the other endpoint, and independently of the other endpoint.

It is also understood that there are a number of values disclosed herein, and that each value is also herein disclosed as “about” or “approximately” that particular value in addition to the value itself. For example, if the value “10” is disclosed, then “approximately 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed then “less than or equal to 10” as well as “greater than or equal to 10” is also disclosed. Definitions

The terms “extracellular matrix”, “ECM” and “ECM material” are used interchangeably herein, and mean and include a collagen-rich substance that is found in between cells in mammalian tissue, and any material processed therefrom, e.g. decellularized ECM. According to the invention, the ECM material can be derived from a variety of mammalian tissue sources, including, without limitation, small intestine submucosa (SIS), urinary bladder submucosa (UBS), stomach submucosa (SS), central nervous system tissue, epithelium of mesodermal origin, i.e. mesothelial tissue, dermal extracellular matrix, subcutaneous extracellular matrix, gastrointestinal extracellular matrix, i.e. large and small intestines, tissue surrounding growing bone, placental extracellular matrix, ornomentum extracellular matrix, cardiac extracellular matrix, e.g., pericardium and/or myocardium, kidney extracellular matrix, pancreas extracellular matrix, lung extracellular matrix, and combinations thereof. The ECM material can also comprise collagen from mammalian sources.

The terms “urinary bladder submucosa (UBS)”, “small intestine submucosa (SIS)” and “stomach submucosa (SS)” also mean and include any UBS and/or SIS and/or SS material that includes the tunica mucosa (which includes the transitional epithelial layer and the tunica propria), submucosal layer, one or more layers of muscularis, and adventitia (a loose connective tissue layer) associated therewith.

The ECM material can also be derived from basement membrane of mammalian tissue/organs, including, without limitation, urinary basement membrane (UBM), liver basement membrane (LBM), and amnion, chorion, allograft pericardium, allograft acellular dermis, amniotic membrane, Wharton's jelly, and combinations thereof.

Additional sources of mammalian basement membrane include, without limitation, spleen, lymph nodes, salivary glands, prostate, pancreas and other secreting glands.

The ECM material can also be derived from other sources, including, without limitation, collagen from plant sources and synthesized extracellular matrices, i.e. cell cultures.

The term “angiogenesis”, as used herein, means a physiologic process involving the growth of new blood vessels from pre-existing blood vessels.

The term “neovascularization”, as used herein, means and includes the formation of functional vascular networks that can be perfused by blood or blood components. Neovascularization includes angiogenesis, budding angiogenesis, intussuceptive angiogenesis, sprouting angiogenesis, therapeutic angiogenesis and vasculogenesis.

The term “Artelon”, as used herein, means the knitted poly(urethane urea) material distributed by Artimplant AB in Goteborg, Sweden.

The terms “biologically active agent” and “biologically active composition” are used interchangeably herein, and mean and include agent that induces or modulates a physiological or biological process, or cellular activity, e.g., induces proliferation, and/or growth and/or regeneration of tissue.

The terms “biologically active agent” and “biologically active composition” thus mean and include, without limitation, the following growth factors: platelet derived growth factor (PDGF), epidermal growth factor (EGF), transforming growth factor alpha (TGF-alpha), transforming growth factor beta (TGF-beta), fibroblast growth factor-2 (FGF-2), basic fibroblast growth factor (bFGF), vascular epithelial growth factor (VEGF), hepatocyte growth factor (HGF), insulin-like growth factor (IGF), nerve growth factor (NGF), platlet derived growth factor (PDGF), tumor necrosis factor alpha (TNA-alpha), and placental growth factor (PLGF).

The terms “biologically active agent” and “biologically active composition” also mean and include, without limitation, human embryonic stern cells, fetal cardiomyocytes, myofibroblasts, mesenchymal stem cells, autotransplated expanded cardiomyocytes, adipocytes, totipotent cells, pluripotent cells, blood stem cells, myoblasts, adult stem cells, bone marrow cells, mesenchymal cells, embryonic stern cells, parenchymal cells, epithelial cells, endothelial cells, mesothelial cells, fibroblasts, osteoblasts, chondrocytes, exogenous cells, endogenous cells, stem cells, hematopoietic stern cells, bone-marrow derived progenitor cells, myocardial cells, skeletal cells, fetal cells, undifferentiated cells, multi-potent progenitor cells, unipotent progenitor cells, monocytes, cardiac myoblasts, skeletal myoblasts, macrophages, capillary endothelial cells, xenogenic cells, allogenic cells, and post-natal stem cells.

The terms “biologically active agent” and “biologically active composition” also mean and include, without limitation, the following biologically active agents (referred to interchangeably herein as a “protein”, “peptide” and “polypeptide”): collagen (types I-V), proteoglycans, glycosaminoglycans (GAGs), glycoproteins, growth factors, cytokines, cell-surface associated proteins, cell adhesion molecules (CAM), angiogenic growth factors, endothelial ligands, matrikines, cadherins, immuoglobins, fibril collagens, non-fibrallar collagens, basement membrane collagens, multiplexins, small-leucine rich proteoglycans, decorins, biglycans, fibromodulins, keratocans, lumicans, epiphycans, heparin sulfate proteoglycans, perlecans, agrins, testicans, syndecans, glypicans, serglycins, selectins, lecticans, aggrecans, versicans, neurocans, brevicans, cytoplasmic domain-44 (CD-44), macrophage stimulating factors, amyloid precursor proteins, heparins, chondroitin sulfate B (dermatan sulfate), chondroitin sulfate A, heparin sulfates, hyaluronic acids, fibronectins, tenascins, elastins, fibrillins, laminins, nidogen/enactins, fibulin I, finulin II, integrins, transmembrane molecules, thrombospondins, ostepontins, and angiotensin converting enzymes (ACE).

The terms “pharmacological agent”, “active agent”, “drug” and “active agent formulation” are used interchangeably herein, and mean and include an agent, drug, compound, composition of matter or mixture thereof, including its formulation, which provides some therapeutic, often beneficial, effect. This includes any physiologically or pharmacologically active substance that produces a localized or systemic effect or effects in animals, including warm blooded mammals, humans and primates; avians; domestic household or farm animals, such as cats, dogs, sheep, goats, cattle, horses and pigs; laboratory animals, such as mice, rats and guinea pigs; fish; reptiles; zoo and wild animals; and the like.

The terms “pharmacological agent”, “active agent”, “drug” and “active agent formulation” thus mean and include, without limitation, antibiotics, anti-arrhythmic agents, anti-viral agents, analgesics, steroidal anti-inflammatories, non-steroidal anti-inflammatories, anti-neoplastics, anti-spasmodics, modulators of cell-extracellular matrix interactions, proteins, hormones, growth factors, matrix metalloproteinases (MMPS), enzymes and enzyme inhibitors, anticoagulants and/or antithrombic agents, DNA, RNA, modified DNA and RNA, NSAIDs, inhibitors of DNA, RNA or protein synthesis, polypeptides, oligonucleotides, polynucleotides, nucleoproteins, compounds modulating cell migration, compounds modulating proliferation and growth of tissue, and vasodilating agents.

The terms “pharmacological agent”, “active agent”, “drug” and “active agent formulation” thus include, without limitation, atropine, tropicamide, dexamethasone, dexamethasone phosphate, betamethasone, betamethasone phosphate, prednisolone, triamcinolone, triamcinolone acetonide, fluocinolone acetonide, anecortave acetate, budesonide, cyclosporine, FK-506, rapamycin, ruboxistaurin, midostaurin, flurbiprofen, suprofen, ketoprofen, diclofenac, ketorolac, nepafenac, lidocaine, neomycin, polymyxin b, bacitracin, gramicidin, gentamicin, oyxtetracycline, ciprofloxacin, ofloxacin, tobramycin, amikacin, vancomycin, cefazolin, ticarcillin, chloramphenicol, miconazole, itraconazole, trifluridine, vidarabine, ganciclovir, acyclovir, cidofovir, ara-amp, foscarnet, idoxuridine, adefovir dipivoxil, methotrexate, carboplatin, phenylephrine, epinephrine, dipivefrin, timolol, 6-hydroxydopamine, betaxolol, pilocarpine, carbachol, physostigmine, demecarium, dorzolamide, brinzolamide, latanoprost, sodium hyaluronate, insulin, verteporfin, pegaptanib, ranibizumab, and other antibodies, antineoplastics, anti VGEFs, ciliary neurotrophic factor, brain-derived neurotrophic factor, bFGF, Caspase-1 inhibitors, Caspase-3 inhibitors, α-Adrenoceptors agonists, NMDA antagonists, Glial cell line-derived neurotrophic factors (GDNF), pigment epithelium-derived factor (PEDF), and NT-3, NT-4, NGF, IGF-2.

The terms “pharmacological agent”, “active agent”, “drug” and “active agent formulation” further mean and include the following Class I-Class V antiarrhythmic agents: (Class Ia) quinidine, procainamide and disopyramide; (Class Ib) lidocaine, phenytoin and mexiletine; (Class Ic) flecainide, propafenone and moricizine; (Class II) propranolol, esmolol, timolol, metoprolol and atenolol; (Class III) amiodarone, sotalol, ibutilide and dofetilide; (Class IV) verapamil and diltiazem) and (Class V) adenosine and digoxin.

The terms “pharmacological agent”, “active agent”, “drug” and “active agent formulation” further mean and include, without limitation, the following antiobiotics: aminoglycosides, cephalosporins, chloramphenicol, clindamycin, erythromycins, fluoroquinolones, macrolides, azolides, metronidazole, penicillins, tetracyclines, trimethoprim-sulfamethoxazole and vancomycin.

The terms “pharmacological agent”, “active agent”, “drug” and “active agent formulation” further include, without limitation, the following steroids: andranes (e.g., testosterone), cholestanes, cholic acids, corticosteroids (e.g., dexamethasone), estraenes (e.g., estradiol) and pregnanes (e.g., progesterone).

The terms “pharmacological agent”, “active agent”, “drug” and “active agent formulation” can further include one or more classes of narcotic analgesics, including, without limitation, morphine, codeine, heroin, hydromorphone, levorphanol, meperidine, methadone, oxycodone, propoxyphene, fentanyl, methadone, naloxone, buprenorphine, butorphanol, nalbuphine and pentazocine.

The terms “pharmacological agent”, “active agent”, “drug” and “active agent formulation” can further include one or more classes of topical or local anesthetics, including, without limitation, esters, such as benzocaine, chloroprocaine, cocaine, cyclomethycaine, dimethocaine/larocaine, piperocaine, propoxycaine, procaine/novacaine, proparacaine, and tetracaine/amethocaine. Local anesthetics can also include, without limitation, amides, such as articaine, bupivacaine, cinchocaine/dibucaine, etidocaine, levobupivacaine, lidocaine/lignocaine, mepivacaine, prilocaine, ropivacaine, and trimecaine. Local anesthetics can further include combinations of the above from either amides or esters.

The terms “pharmacological agent”, “active agent”, “drug” and “active agent formulation” can further include one or more classes of cytotoxic anti-neoplastic agents or chemotherapy agents, including, without limitation, alkylating agents, cisplatin, carboplatin, oxaliplatin, mechlorethamine, cyclophosphamide, chlorambucil, and ifosfamide.

Chemotherapy agents can also include, without limitation, antimetabolites, such as purine analogues, pyrimidine analogues and antifolates, plant alkaloids, such as vincristine, vinblastine, vinorelbine, vindesine, podophyllotoxin, etoposide and teniposide, taxanes, such as paclitaxel and docetaxel, topoisomerase inhibitors, such as irinotecan, topotecan, amsacrine, etoposide, etoposide phosphate and teniposide, cytotoxic antibiotics, such as actinomyocin, bleomycin, plicamycin, mytomycin and anthracyclines, such as doxorubicin, daunorubicin, valrubicin, idarubicin, epirubicin, and antibody treatments, such as abciximab, adamlimumab, alamtuzumab, basiliximab, belimumab, bevacizumab, brentuximab vedotin, canakinumab, cetuximab, certolizumab pego, daclizumab, denosumab, eculizumab, efalizumab, gemtuzumab, golimumab, ibritumomab tiuxetan, infliximab, ipilimumab, muromonab-CD3, natalizumab, ofatumumab, omalizumab, palivizumab, panitumumab, ranibizumab, rituximab, tocilizumab (atlizumab), tositumomab and trastuzumab.

The terms “anti-inflammatory” and “anti-inflammatory agent” are also used interchangeably herein, and mean and include a “pharmacological agent” and/or “active agent formulation”, which, when a therapeutically effective amount is administered to a subject, prevents or treats bodily tissue inflammation i.e. the protective tissue response to injury or destruction of tissues, which serves to destroy, dilute, or wall off both the injurious agent and the injured tissues.

Anti-inflammatory agents thus include, without limitation, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide di sodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, decanoate, deflazacort, delatestryl, depo-testosterone, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, mesterolone, methandrostenolone, methenolone, methenolone acetate, methylprednisolone suleptanate, momiflumate, nabumetone, nandrolone, naproxen, naproxen sodium, naproxol, nimazone, olsalazine sodium, orgotein, orpanoxin, oxandrolane, oxaprozin, oxyphenbutazone, oxymetholone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, stanozolol, sudoxicam, sulindac, suprofen, talmetacin, talniflumate, talosalate, tebufelone, tenidap, tenidap sodium, tenoxicam, tesicam, tesimide, testosterone, testosterone blends, tetrydamine, tiopinac, tixocortol pivalate, tolmetin, tolmetin sodium, triclonide, triflumidate, zidometacin, and zomepirac sodium.

The term “pharmacological composition”, as used herein, means and includes a composition comprising a “pharmacological agent” and/or a “biologically active agent” and/or any additional agent or component identified herein.

The term “therapeutically effective”, as used herein, means that the amount of the “pharmacological agent” and/or “biologically active agent” and/or “pharmacological composition” administered is of sufficient quantity to ameliorate one or more causes, symptoms, or sequelae of a disease or disorder. Such amelioration only requires a reduction or alteration, not necessarily elimination, of the cause, symptom, or sequelae of a disease or disorder.

The terms “patient” and “subject” are used interchangeably herein, and mean and include warm blooded mammals, humans and primates; avians; domestic household or farm animals, such as cats, dogs, sheep, goats, cattle, horses and pigs; laboratory animals, such as mice, rats and guinea pigs; fish; reptiles; zoo and wild animals; and the like.

The term “comprise” and variations of the term, such as “comprising” and “comprises,” means “including, but not limited to” and is not intended to exclude, for example, other additives, components, integers or steps.

The following disclosure is provided to further explain in an enabling fashion the best modes of performing one or more embodiments of the present invention. The disclosure is further offered to enhance an understanding and appreciation for the inventive principles and advantages thereof, rather than to limit in any manner the invention. The invention is defined solely by the appended claims including any amendments made during the pendency of this application and all equivalents of those claims as issued.

As stated above, the present invention is directed to reinforced prosthetic atrioventricular tissue valves that can be readily employed to selectively replace diseased or defective mitral and tricuspid valves, and methods for attaching same to cardiovascular structures and tissue.

As discussed in detail herein, the reinforced prosthetic atrioventricular valves comprise continuous tubular members having first (or proximal) and second (or distal) ends, such as the tubular prosthetic valves disclosed in Co-Pending U.S. application Ser. Nos. 13/480,347 and 13/480,324; which are incorporated by reference herein.

In a preferred embodiment, the tubular members include a reinforcement member (i.e. tubular member constructs) that is designed and configured to enhance the structural integrity of the tubular member and, hence, prosthetic atrioventricular tissue valve formed therefrom, and facilitate secure engagement of the prosthetic valve to cardiovascular structures, e.g., selective papillary muscles, ventricles, etc., and/or cardiovascular tissue.

In a preferred embodiment of the invention, the prosthetic atrioventricular valves (or tubular member constructs) are capable of transitioning from a pre-deployment configuration, wherein the prosthetic valves are capable of being positioned within a cardiovascular vessel, to a post-deployment configuration, wherein the prosthetic valves are disposed proximate host tissue of the vessel.

As indicated above, in some embodiments of the invention, the reinforcement member includes anchoring means that is designed and configured to position the prosthetic valves proximate host tissue of a vessel, and maintain contact therewith for a pre-determined period of time. Suitable anchoring means are disclosed in Co-pending U.S. application Ser. Nos. 13/686,131, 13/782,024 and 13/804,683; which are incorporated by reference herein in their entirety.

In some embodiments of the invention, the reinforcement member comprises a plurality of linear members.

In some embodiments, the reinforcement member comprises a multiple-link member.

In some embodiments, the multiple-link member comprises an integral member.

In some embodiments, each link of the multiple-link reinforcement member comprises a separate member.

In a preferred embodiment of the invention, the reinforcement member comprises a biocompatible material. In some embodiments, the reinforcement member comprises a biocompatible and biodegradable material.

In some embodiments, the reinforcement member comprises magnesium.

In some embodiments, the reinforcement member comprises stainless steel.

In some embodiments, the reinforcement member comprises a cobalt-chrome nickel alloy.

In some embodiments, the reinforcement member comprises Nitinol®.

In some embodiments of the invention, the metal reinforcement members include an immunomodulating compound.

In some embodiments, the immunomodulating compound comprises a polysaccharide, including, without limitation, GAGs, dextrans, alginate and chitosan.

In some embodiments, immunomodulating compound comprises a polymeric material, including, without limitation, high molecular weight hyaluronic acid (HMW-HA).

In some embodiments of the invention, the reinforcement member comprises an ECM material.

In some embodiments of the invention, the reinforcement member comprises a cross-linked ECM material.

In some embodiments of the invention, the reinforcement member comprises a polymeric material.

In some embodiments, the polymeric material comprises Dacron, polyether ether ketone (PEEK), and like materials

In some embodiments of the invention, the reinforcement member comprises Artelon®.

In some embodiments of the invention, each link of the multiple-link reinforcement member is formed from the same material.

In some embodiments, each link of the multiple-link reinforcement member is formed from a different material.

In some embodiments of the invention, at least a portion of the reinforcement member is encased in an ECM material.

In some embodiments, the entire reinforcement member is encased in an ECM material.

According to the invention, the ECM encasement material can be disposed on the reinforcement member via various conventional means, including, without limitation, wrapping the reinforcement member with an ECM material, i.e. sheet, dipping the reinforcement member in a fluidized ECM material and/or spraying the reinforcement member with an ECM material.

According to the invention, the tubular members can comprise various biocompatible materials.

In some embodiments of the invention, the tubular members comprise a biocompatible polymeric material.

In some embodiments, the tubular members comprise an ECM material.

According to the invention, the ECM material referenced above can be derived from various mammalian tissue sources and methods for preparing same, such as disclosed in U.S. Pat. Nos. 7,550,004, 7,244,444, 6,379,710, 6,358,284, 6,206,931, 5,733,337 and 4,902,508 and U.S. application Ser. No. 12/707,427; which are incorporated by reference herein in their entirety.

In a preferred embodiment, the mammalian tissue sources include, without limitation, small intestine submucosa (SIS), urinary bladder submucosa (UBS), stomach submucosa (SS), central nervous system tissue, epithelium of mesodermal origin, i.e. mesothelial tissue, dermal extracellular matrix, subcutaneous extracellular matrix, gastrointestinal extracellular matrix, i.e. large and small intestines, tissue surrounding growing bone, placental extracellular matrix, ornomentum extracellular matrix, cardiac extracellular matrix, e.g., pericardium and/or myocardium, kidney extracellular matrix, pancreas extracellular matrix, lung extracellular matrix, and combinations thereof. The ECM material can also comprise collagen from mammalian sources.

As stated above, in some embodiments of the invention, the tubular members (or a portion thereof) and/or reinforcement member encasement material(s) include at least one additional biologically active agent or composition, i.e. an agent that induces or modulates a physiological or biological process, or cellular activity, e.g., induces proliferation, and/or growth and/or regeneration of tissue.

Suitable biologically active agents include any of the aforementioned biologically active agents, including, without limitation, the aforementioned cells and proteins.

In some embodiments, the tubular members (or a portion thereof) and/or reinforcement member encasement material(s) include at least one pharmacological agent or composition (or drug), i.e. an agent or composition that is capable of producing a desired biological effect in vivo, e.g., stimulation or suppression of apoptosis, stimulation or suppression of an immune response, etc.

Suitable pharmacological agents and compositions include any of the aforementioned agents, including, without limitation, antibiotics, anti-viral agents, analgesics, steroidal anti-inflammatories, non-steroidal anti-inflammatories, anti-neoplastics, anti-spasmodics, modulators of cell-extracellular matrix interactions, proteins, hormones, enzymes and enzyme inhibitors, anticoagulants and/or antithrombic agents, DNA, RNA, modified DNA and RNA, NSAIDs, inhibitors of DNA, RNA or protein synthesis, polypeptides, oligonucleotides, polynucleotides, nucleoproteins, compounds modulating cell migration, compounds modulating proliferation and growth of tissue, and vasodilating agents.

In some embodiments of the invention, the pharmacological agent comprises an anti-inflammatory agent.

In some embodiments of the invention, the pharmacological agent comprises a statin, i.e. a HMG-CoA reductase inhibitor. According to the invention, suitable statins include, without limitation, atorvastatin (Lipitor®), cerivastatin, fluvastatin (Lescol®), lovastatin (Mevacor®, Altocor®, Altoprev®), mevastatin, pitavastatin (Livalo®, Pitava®), pravastatin (Pravachol®, Selektine®, Lipostat®), rosuvastatin (Crestor®), and simvastatin (Zocor®, Lipex®). Several actives comprising a combination of a statin and another agent, such as ezetimbe/simvastatin (Vytorin®), are also suitable.

In some embodiments of the invention, the pharmacological agent comprises chitosan.

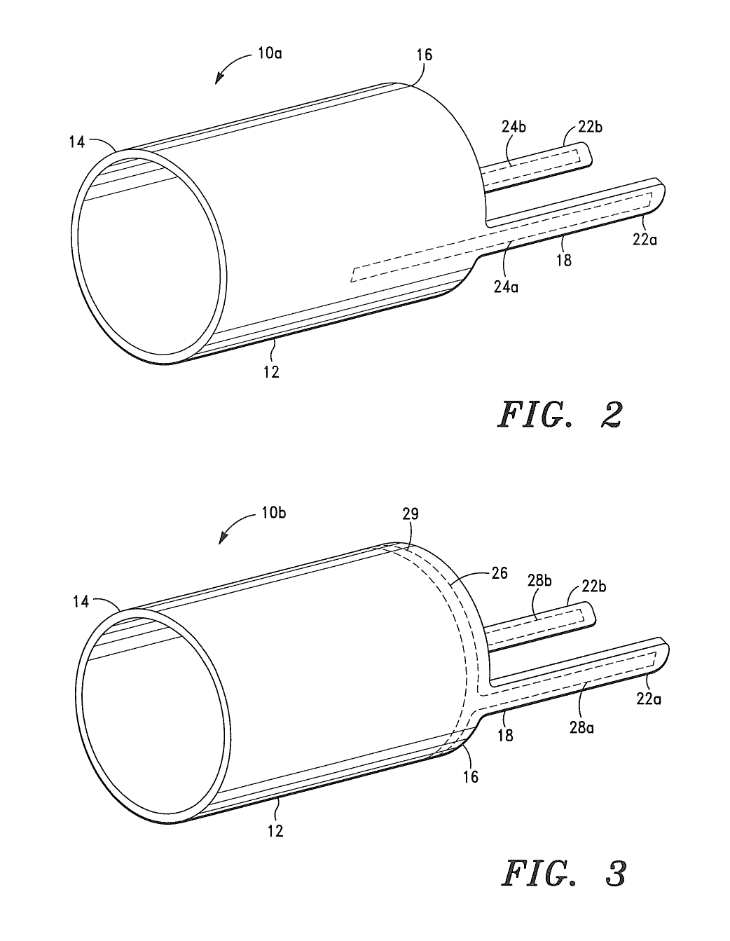

Referring now to FIG. 2, a first embodiment of a reinforced prosthetic atrioventricular tissue valve will be described in detail.

As illustrated in FIG. 2, the prosthetic atrioventricular tissue valve 10a comprises a continuous tubular member 12 having first or “proximal” and second or “distal” ends 14, 16. In some embodiments of the invention, the valve 10a includes at least one internal leaflet, such as disclosed in Co-Pending U.S. application Ser. Nos. 13/804,683 and 13/782,289. In some embodiments, the tubular member 12 includes a leaflet forming interior surface, such as disclosed in Co-Pending U.S. application Ser. Nos. 13/480,324 and 13/480,347.

According to the invention, the tubular member 12 can comprise various biocompatible materials.

In some embodiments of the invention, the tubular member 12 comprises a biocompatible polymeric material. In some embodiments, the polymeric material comprises Dacron, polyether ether ketone (PEEK), and like materials.

In a preferred embodiment, the tubular member 12 comprises an ECM material.

According to the invention, the ECM material can be derived from various mammalian tissue sources including, without limitation, small intestine submucosa (SIS), urinary bladder submucosa (UBS), stomach submucosa (SS), central nervous system tissue, epithelium of mesodermal origin, i.e. mesothelial tissue, dermal extracellular matrix, subcutaneous extracellular matrix, gastrointestinal extracellular matrix, i.e. large and small intestines, tissue surrounding growing bone, placental extracellular matrix, ornamentum extracellular matrix, cardiac extracellular matrix, e.g., pericardium and/or myocardium, kidney extracellular matrix, pancreas extracellular matrix, lung extracellular matrix, and combinations thereof. The ECM material can also comprise collagen from mammalian sources.

In some embodiments of the invention, the tubular member 12 includes at least one additional biologically active agent or composition, i.e. an agent that induces or modulates a physiological or biological process, or cellular activity, e.g., induces proliferation, and/or growth and/or regeneration of tissue.

Suitable biologically active agents include any of the aforementioned biologically active agents.

In some embodiments, the tubular member 12 includes at least one pharmacological agent or composition (or drug), i.e. an agent or composition that is capable of producing a desired biological effect in vivo, e.g., stimulation or suppression of apoptosis, stimulation or suppression of an immune response, etc.

Suitable pharmacological agents and compositions include any of the aforementioned agents, including, without limitation, antibiotics, anti-viral agents, analgesics, steroidal anti-inflammatories, non-steroidal anti-inflammatories, anti-neoplastics, anti-spasmodics, modulators of cell-extracellular matrix interactions, proteins, hormones, enzymes and enzyme inhibitors, anticoagulants and/or antithrombic agents, DNA, RNA, modified DNA and RNA, NSAIDs, inhibitors of DNA, RNA or protein synthesis, polypeptides, oligonucleotides, polynucleotides, nucleoproteins, compounds modulating cell migration, compounds modulating proliferation and growth of tissue, and vasodilating agents.

In a preferred embodiment, the second or “distal” end 16 of the tubular member 12 includes cardiovascular structure engagement means 18 that is designed and configured to securely engage the member 12 and, hence, prosthetic atrioventricular tissue valve 10a formed therefrom to cardiovascular structures, such as selective papillary muscles, and/or cardiovascular tissue.

As illustrated in FIG. 2, in some embodiments, the cardiovascular structure engagement means 18 comprises a pair of cardiovascular structure engagement members 22a, 22b, which extend from the tubular member 12 to mimic the chordae tendinae. According to the invention, the cardiovascular structure engagement members 22a, 22b can be disposed at various positions about the periphery of the distal end 16 of the tubular member 12.

In some embodiments of the invention, wherein the prosthetic atrioventricular tissue valve 10a is employed to replace a mitral valve, the cardiovascular structure engagement members 22a, 22b are spaced at approximately 0° and 120°.

According to the invention, the cardiovascular structure engagement members 22a, 22b can also have various predetermined lengths to accommodate attachment to desired cardiovascular structures, e.g., selective papillary muscles.

In a preferred embodiment of the invention, the cardiovascular structure engagement members 22a, 22b include reinforcement members 24a, 24b. As illustrated in FIG. 2, the reinforcement members 24a, 24b preferably extend throughout the length of the engagement members 22a, 22b. In a preferred embodiment, the reinforcement members 24a, 24b also extend over a predetermined length of the tubular member 12, as shown in FIG. 2.

According to the invention, the reinforcement members 24a, 24b can comprise various forms and materials. In a preferred embodiment, the reinforcement members 24a, 24b comprise a ribbon or chord (or braid) of biocompatible material.

In some embodiments, the reinforcement members 24a, 24b comprise a cross-linked ECM material.

In some embodiments, the reinforcement members 24a, 24b comprise magnesium or stainless steel.

In some embodiments, the reinforcement members 24a, 24b comprise a polymeric material.

In some embodiments, the reinforcement members 24a, 24b comprise Artelong.

As indicated above, in a preferred embodiment of the invention, the reinforcement members 24a, 24b are encased in at least one of the aforementioned ECM materials.

According to the invention, the reinforcement members 24a, 24b can be disposed proximate (or on) the outer surface of the tubular member 12 and cardiovascular structure engagement members 22a, 22b or incorporated in the tubular member 12 and cardiovascular structure engagement members 22a, 22b (i.e. encased by the tubular member 12 and reinforcement members 24a, 24b material), as shown in FIG. 2.

Referring now to FIG. 3, there is shown another embodiment of a reinforced prosthetic atrioventricular tissue valve 10b of the invention. As illustrated in FIG. 3, the reinforced prosthetic valve 10b similarly comprises a continuous tubular member 12 having proximal and distal ends 14, 16.

According to the invention, the tubular member 12 can similarly comprise any of the aforementioned materials and can include one or more of the additional biologically active and/or pharmacological agents.

The distal end 16 of the tubular member 12 similarly includes cardiovascular structure engagement means 18, i.e. cardiovascular structure engagement members 22a, 22b, that is designed and configured to securely engage the member 12 and, hence, reinforced prosthetic atrioventricular tissue valve 10b formed therefrom to cardiovascular structures, e.g. selective papillary muscles, and/or cardiovascular tissue.

In a preferred embodiment, the cardiovascular structure engagement members 22a, 22b are similarly reinforced via a reinforcement member 26, having elongated members 28a, 28b that are preferably similar to reinforcement members 24a, 24b. However, in this embodiment, the reinforcement member 26 further includes an annular ring 29.

According to the invention, the reinforcement member 26 can similarly comprise any of the aforementioned materials. In a preferred embodiment, the reinforcement member 26 is also encased in at least one of the aforementioned ECM materials.

The elongated members 28a, 28b and annular component 29 can also comprise an integral member or separate components (or members).

According to the invention, the “reinforced” cardiovascular structure engagement members 22a, 22b discussed above significantly enhance the strength and, hence, structural integrity of the prosthetic valves 10a, 10b when attached to a cardiovascular structure; particularly, the papillary muscles. The cardiovascular structure engagement members 22a, 22b also preserve the structural integrity of the papillary muscles when attached thereto.

The “reinforced” cardiovascular structure engagement members 22a, 22b, when attached to a cardiovascular structure, thus, significantly reduce the risk of suture failure and rupture of the engaged cardiovascular structure, e.g., papillary muscles, and prosthetic valve tissue proximate thereto.

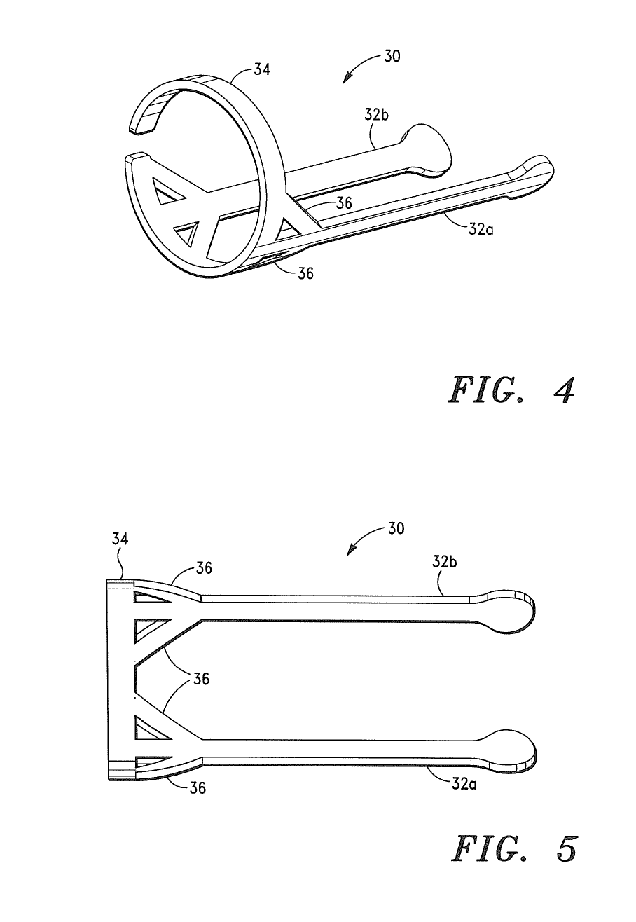

Referring now to FIGS. 4-7, another embodiment of a prosthetic valve reinforcement member and tubular member construct, i.e. reinforced prosthetic tissue valve, formed therewith will be described in detail.

Referring first to FIGS. 4 and 5, in this embodiment the prosthetic valve includes a reinforcement member 30. In a preferred embodiment, the reinforcement member 30 comprises a multiple-link member.

As illustrated in FIGS. 4 and 5, the member 30 includes at least one, more preferably two (2) elongated members 32a, 32b, an annular ring 34, and structural support members 36. According to the invention, the annular ring 34 can comprise a continuous member or a non-continuous member, as shown in FIG. 4.

According to the invention, the elongated members 32a, 32b, annular ring 34 and structural support members 36 can comprise an integral member, as shown in FIG. 5, or separate members.

The elongated members 32a, 32b, annular ring 34 and structural support members 36 can also comprise any of the aforementioned reinforcement member materials. The elongated members 32a, 32b, annular ring 34 and structural support members 36 can also comprise the same material or different materials.

In a preferred embodiment of the invention, the elongated members 32a, 32b and/or annular ring 34 and/or structural support members 36 are also encased in an ECM material, as described above.

According to the invention, the ECM encasement material can also include at least one of the aforementioned biologically active and/or pharmacological agents.

According to the invention, the elongated members 32a, 32b can be disposed at various positions about the periphery of the annular ring 34.

In some embodiments of the invention, wherein a reinforced prosthetic atrioventricular tissue valve of the invention (such as prosthetic valve 10c, discussed below) is employed to replace a mitral valve, the elongated members 32a, 32b are preferably spaced at approximately 0° and 120° on the annular ring 34.

According to the invention, the elongated members 32a, 32b can also have various predetermined lengths to accommodate attachment to desired cardiovascular structures, e.g., selective papillary muscles, and/or tissue.

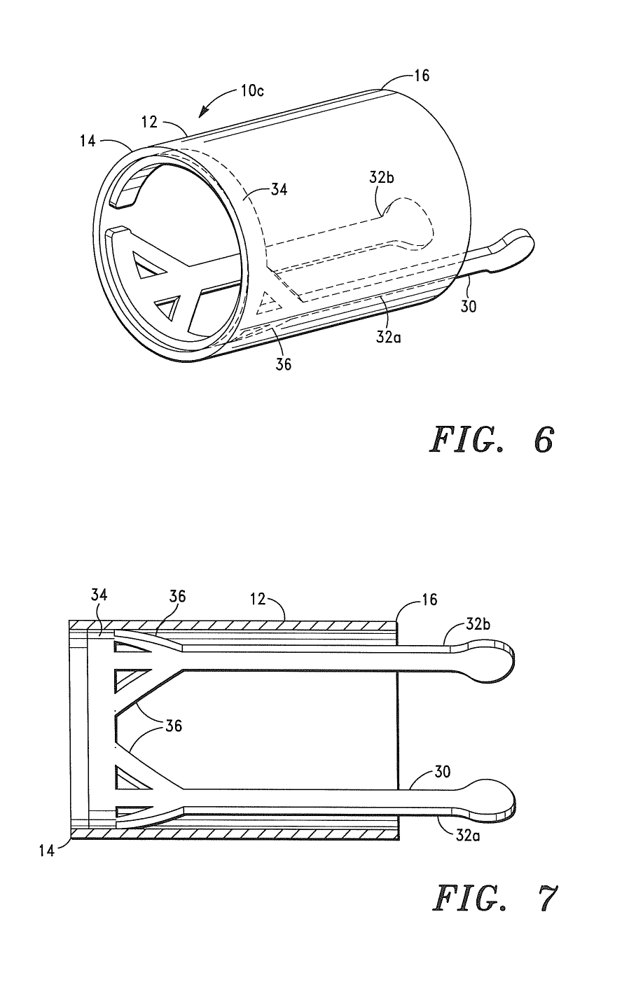

Referring now to FIGS. 6 and 7, one embodiment of a tubular construct, i.e. reinforced prosthetic tissue valve, formed with the reinforcement member 30 will be described in detail.

As illustrated in FIG. 6, the prosthetic valve 10c similarly includes a continuous tubular member 12 having proximal and distal ends 14, 16.

In some embodiments of the invention, the tubular member 12 includes at least one internal leaflet, such as disclosed in Co-Pending U.S. application Ser. Nos. 13/804,683 and 13/782,289. In some embodiments, the tubular member 12 includes a leaflet forming interior surface, such as disclosed in Co-Pending U.S. application Ser. Nos. 13/480,324 and 13/480,347.

According to the invention, the tubular member 12 can similarly comprise any of the aforementioned materials. In a preferred embodiment, the tubular member 12 comprises an ECM material, e.g. mesothelial tissue.

The ECM material can also include at least one of the aforementioned biologically active and/or pharmacological agents, e.g., a statin or an antibiotic.

In some embodiments of the invention, at least the annular ring 34 of the reinforcement member 30 is embedded in the tubular member 12 material, e.g. sandwiched within ECM sheets, wherein at least a portion of the elongated members 32a, 32b extend or project out of the distal end 16 of the tubular member 12, as shown in FIGS. 6 and 7.

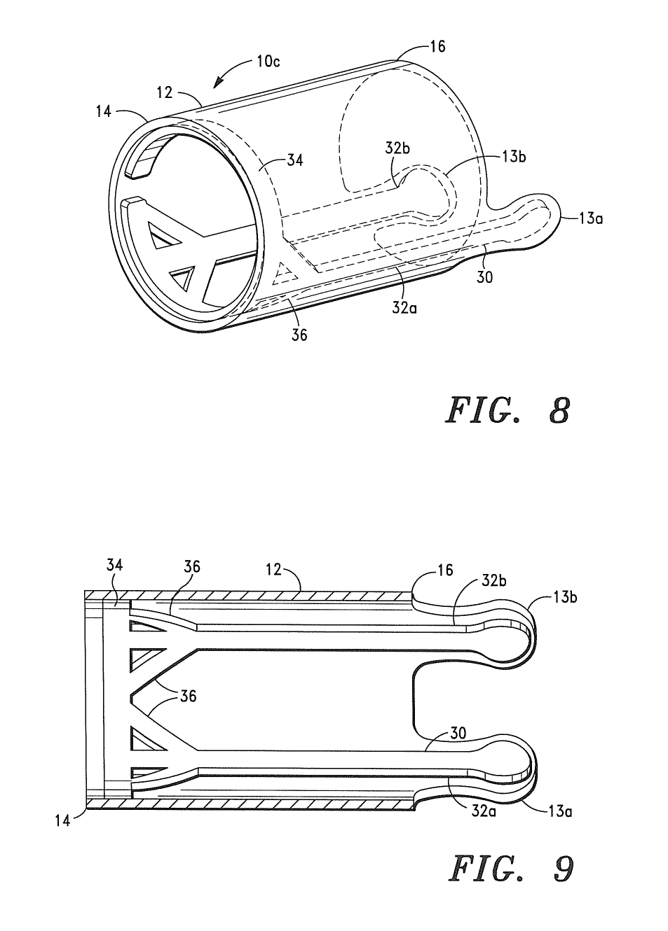

In some embodiments, the entire reinforcement member 30 is embedded in the tubular member 12 material, wherein the elongated members 32a, 32b are encased in tubular member extensions 13a, 13b, as shown in FIGS. 8 and 9.

In an alternative envisioned embodiment, the reinforcement member 30 is disposed on the outer surface of the tubular member.

According to the invention, the reinforcement member 30 enhances the multi-dimensional flexure of the prosthetic valve 10c during cardiac cycles. The reinforcement member 30 also significantly enhances the strength and, hence, structural integrity of the prosthetic valve 10c when attached to a cardiovascular structure; particularly, the papillary muscles. The reinforcement member 30 also preserves the structural integrity of the papillary muscles when attached thereto.

The reinforcement member 30, when attached to a cardiovascular structure, thus, significantly reduces the risk of suture failure and rupture of the engaged cardiovascular structure, e.g., papillary muscles, and prosthetic valve tissue proximate thereto.

In a preferred embodiment of the invention, the prosthetic atrioventricular valve 10c is also capable of transitioning from a pre-deployment configuration, wherein the prosthetic valve 10c is capable of being positioned within a cardiovascular vessel, to a post-deployment configuration, wherein the prosthetic valve 10c is disposed proximate host tissue of the vessel.

In some embodiments of the invention, the reinforcement member 30 also includes anchoring means that designed and configured to position the prosthetic valve 10c proximate desired cardiovascular tissue (or a desired vessel region), and maintain contact therewith for a pre-determined period of time. As indicated above, suitable anchoring means are disclosed in Co-pending U.S. application Ser. Nos. 13/686,131, 13/782,024 and 13/804,683.

In some embodiments, the anchoring means includes a plurality of barbs or microneedles that extend from the annular ring 34.

As will readily be appreciated by one having ordinary skill in the art, the present invention provides numerous advantages compared to prior art prosthetic valves. Among the advantages are the following:
The provision of reinforced prosthetic atrioventricular tissue valves and methods for attaching same to cardiovascular structures and/or tissue, which enhance the structural integrity of the valve.
The provision of reinforced prosthetic atrioventricular tissue valves and methods for attaching same to cardiovascular structures and/or tissue, which preserve the structural integrity of the cardiovascular structure(s) when attached thereto.
The provision of prosthetic atrioventricular tissue valves having means for secure, reliable, and consistently highly effective attachment to cardiovascular structures and/or tissue.
The provision of improved methods for securely attaching prosthetic atrioventricular valves to cardiovascular structures and/or tissue.
The provision of reinforced prosthetic atrioventricular tissue valves that induce host tissue proliferation, bioremodeling and regeneration of new tissue and tissue structures with site-specific structural and functional properties.
The provision of reinforced prosthetic atrioventricular tissue valves that are capable of administering a pharmacological agent to host tissue and, thereby produce a desired biological and/or therapeutic effect.
The provision of reinforced prosthetic atrioventricular tissue valves that exhibit optimum mechanical compatibility with cardiovascular structures.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

What is claimed is:

1. A prosthetic atrioventricular valve, comprising:
a continuous tubular member having proximal and distal ends and a lumen therethrough, said tubular member comprising a first extracellular matrix (ECM) material; and
a reinforcement member disposed in said tubular member lumen and attached to said distal end of said tubular member, said reinforcement member being configured to connect said joined tubular member and reinforcement member to a cardiovascular structure, said reinforcement member comprising at least two elongated linear members, which, when attached to said distal end of said tubular member, project outwardly therefrom.

2. The atrioventricular valve of claim 1, wherein said reinforcement member comprises a biocompatible and biodegradable material.

3. The atrioventricular valve of claim 2, wherein said reinforcement member comprises a metal selected from the group consisting of magnesium, stainless steel and Nitinol®.

4. The atrioventricular valve of claim 2, wherein said reinforcement member comprises a second ECM material.

5. The atrioventricular valve of claim 4, wherein said ECM material comprises a cross-linked ECM material.

6. The atrioventricular valve of claim 1, wherein said first ECM material is derived from a mammalian tissue source selected from the group consisting of small intestine submucosa, urinary bladder submucosa, stomach submucosa, central nervous system tissue, epithelium of mesodeimal origin, dermal extracellular matrix, subcutaneous extracellular matrix, gastrointestinal extracellular matrix, placental extracellular matrix, ornomentum extracellular matrix, cardiac extracellular matrix, kidney extracellular matrix, pancreas extracellular matrix, lung extracellular matrix, and combinations thereof.

7. The atrioventricular valve of claim 4, wherein said second ECM material is derived from a mammalian tissue source selected from the group consisting of small intestine submucosa, urinary bladder submucosa, stomach submucosa, central nervous system tissue, epithelium of mesodermal origin, dermal extracellular matrix, subcutaneous extracellular matrix, gastrointestinal extracellular matrix, placental extracellular matrix, ornomentum extracellular matrix, cardiac extracellular matrix, kidney extracellular matrix, pancreas extracellular matrix, lung extracellular matrix, and combinations thereof.

8. The atrioventricular valve of claim 6, wherein said first ECM material further comprises an exogenously added biologically active agent.

9. The atrioventricular valve of claim 6, wherein said biologically active agent comprises a growth factor selected from the group consisting of a platelet derived growth factor (PDGF), epidermal growth factor (EGF), transforming growth factor alpha (TGF-alpha), transforming growth factor beta (TGF-beta), fibroblast growth factor-2 (FGF-2), basic fibroblast growth factor (bFGF), vascular epithelial growth factor (VEGF), hepatocyte growth factor (HGF), insulin-like growth factor (IGF), nerve growth factor (NGF), platlet derived growth factor (PDGF), tumor necrosis factor alpha (TNA-alpha), and placental growth factor (PLGF).

10. The atrioventricular valve of claim 6, wherein said biologically active agent comprises a cell selected from the group consisting of a human embryonic stem cell, fetal cardiomyocyte, myofibroblast, mesenchymal stem cell, autotransplated expanded cardiomyocyte, adipocyte, totipotent cell, pluripotent cell, blood stem cell, myoblast, adult stem cell, bone marrow cell, parenchymal cell, epithelial cell, endothelial cell, mesothelial cell, fibroblast, osteoblast, chondrocyte, exogenous cell, endogenous cell, hematopoietic stern cell, myocardial cell, skeletal cell, multi-potent progenitor cell, unipotent progenitor cell, macrophage, xenogenic cell and allogenic cell.

11. The atrioventricular valve of claim 6, wherein said first ECM material further comprises a pharmacological agent selected from the group consisting of antibiotics, anti-viral agents, analgesics, steroidal anti-inflammatories, non-steroidal anti-inflammatories, anti-neoplastics, anti-spasmodics, modulators of cell-extracellular matrix interactions, proteins, hormones, enzymes and enzyme inhibitors, anticoagulants and/or antithrombic agents, DNA, RNA, modified DNA and RNA, NSAIDs, inhibitors of DNA, RNA or protein synthesis, polypeptides, oligonucleotides, polynucleotides, nucleoproteins, compounds modulating cell migration, compounds modulating proliferation and growth of tissue, and vasodilating agents.

12. The atrioventricular valve of claim 6, wherein said first ECM material further comprises a HMG-CoA reductase inhibitor selected from the group consisting of atorvastatin, cerivastatin, fluvastatin, lovastatin, mevastatin, pitavastatin, pravastatin, rosuvastatin, and simvastatin.

13. The atrioventricular valve of claim 2, wherein said reinforcement member comprises a polymeric material.

14. The atrioventricular valve of claim 2, wherein at least a first portion of said reinforcement member is encased in a third ECM material, said third ECM material comprising an ECM material derived from a mammalian tissue source selected from the group consisting of small intestine submucosa, urinary bladder submucosa, stomach submucosa, central nervous system tissue, epithelium of mesodermal origin, dermal extracellular matrix, subcutaneous extracellular matrix, gastrointestinal extracellular matrix, placental extracellular matrix, ornomentum extracellular matrix, cardiac extracellular matrix, kidney extracellular matrix, pancreas extracellular matrix, lung extracellular matrix, and combinations thereof.

15. A prosthetic atrioventricular valve, comprising:
a continuous tubular member having proximal and distal ends and a lumen therethrough, said tubular member comprising a first extracellular matrix (ECM) material; and
a reinforcement member comprising a reinforcement ring having at least two elongated linear members, said at least two elongated members having first and second longitudinal axes and said reinforcement ring having a planar axis, said at least two elongated linear members having cardiovascular structure engagement ends that are configured to connect said reinforcement member to a cardiovascular structure, said at least two elongated members being attached to said reinforcement ring, wherein said first and second longitudinal axes of said at least two elongated members are perpendicular to said planar axis of said reinforcement ring,
said reinforcement ring being disposed in said tubular member lumen and attached proximate said proximal end of said tubular member, wherein said at least two elongated linear members extend toward and project outwardly from said distal end of said tubular member.

16. The atrioventricular valve of claim 15, wherein said reinforcement member comprises a biocompatible and biodegradable material.

17. The atrioventricular valve of claim 16, wherein said reinforcement member comprises a metal selected from the group consisting of magnesium, stainless steel and Nitinol®.

18. The atrioventricular valve of claim 17, wherein said reinforcement member comprises Nitinol®.

19. The atrioventricular valve of claim 18, wherein said joined tubular member and reinforcement member are configured to transition from a pre-deployment configuration, wherein said joined tubular member and reinforcement member are capable of being positioned within a cardiovascular vessel, to a post-deployment configuration, wherein said joined tubular member and reinforcement member are disposed proximate host tissue of said vessel by temperature of said host tissue.

20. The atrioventricular valve of claim 16, wherein said reinforcement member comprises a second ECM material.

21. The atrioventricular valve of claim 20, wherein said ECM material comprises a cross-linked ECM material.

22. The atrioventricular valve of claim 15, wherein said first ECM material is derived from a mammalian tissue source selected from the group consisting of small intestine submucosa, urinary bladder submucosa, stomach submucosa, central nervous system tissue, epithelium of mesodermal origin, dermal extracellular matrix, subcutaneous extracellular matrix, gastrointestinal extracellular matrix, placental extracellular matrix, ornomentum extracellular matrix, cardiac extracellular matrix, kidney extracellular matrix, pancreas extracellular matrix, lung extracellular matrix, and combinations thereof.

23. The atrioventricular valve of claim 20, wherein said second ECM material is derived from a mammalian tissue source selected from the group consisting of small intestine submucosa, urinary bladder submucosa, stomach submucosa, central nervous system tissue, epithelium of mesodermal origin, dermal extracellular matrix, subcutaneous extracellular matrix, gastrointestinal extracellular matrix, placental extracellular matrix, ornomentum extracellular matrix, cardiac extracellular matrix, kidney extracellular matrix, pancreas extracellular matrix, lung extracellular matrix, and combinations thereof.

24. The atrioventricular valve of claim 22, wherein said first ECM material further comprises an exogenously added biologically active agent.

25. The atrioventricular valve of claim 24, wherein said biologically active agent comprises a growth factor selected from the group consisting of a platelet derived growth factor (PDGF), epidermal growth factor (EGF), transforming growth factor alpha (TGF-alpha), transforming growth factor beta (TGF-beta), fibroblast growth factor-2 (FGF-2), basic fibroblast growth factor (bFGF), vascular epithelial growth factor (VEGF), hepatocyte growth factor (HGF), insulin-like growth factor (IGF), nerve growth factor (NGF), platlet derived growth factor (PDGF), tumor necrosis factor alpha (TNA-alpha), and placental growth factor (PLGF).

26. The atrioventricular valve of claim 24, wherein said biologically active agent comprises a cell selected from the group consisting of a human embryonic stem cell, fetal cardiomyocyte, myofibroblast, mesenchymal stern cell, autotransplated expanded cardiomyocyte, adipocyte, totipotent cell, pluripotent cell, blood stern cell, myoblast, adult stem cell, bone marrow cell, parenchymal cell, epithelial cell, endothelial cell, mesothelial cell, fibroblast, osteoblast, chondrocyte, exogenous cell, endogenous cell, hematopoietic stem cell, myocardial cell, skeletal cell, multi-potent progenitor cell, unipotent progenitor cell, macrophage, xenogenic cell and allogenic cell.

27. The atrioventricular valve of claim 24, wherein said first ECM material further comprises a pharmacological agent selected from the group consisting of antibiotics, anti-viral agents, analgesics, steroidal anti-inflammatories, non-steroidal anti-inflammatories, anti-neoplastics, anti-spasmodics, modulators of cell-extracellular matrix interactions, proteins, hormones, enzymes and enzyme inhibitors, anticoagulants and/or antithrombic agents, DNA, RNA, modified DNA and RNA, NSAIDs, inhibitors of DNA, RNA or protein synthesis, polypeptides, oligonucleotides, polynucleotides, nucleoproteins, compounds modulating cell migration, compounds modulating proliferation and growth of tissue, and vasodilating agents.

28. The atrioventricular valve of claim 24, wherein said first ECM material further comprises a HMG-CoA reductase inhibitor selected from the group consisting of atorvastatin, cerivastatin, fluvastatin, lovastatin, mevastatin, pitavastatin, pravastatin, rosuvastatin, and simvastatin.

29. The atrioventricular valve of claim 16, wherein said reinforcement member comprises a polymeric material.

30. The atrioventricular valve of claim 16, wherein at least a first portion of said reinforcement member is encased in a third ECM material, said third ECM material comprising an ECM material derived from a mammalian tissue source selected from the group consisting of small intestine submucosa, urinary bladder submucosa, stomach submucosa, central nervous system tissue, epithelium of mesodermal origin, dermal extracellular matrix, subcutaneous extracellular matrix, gastrointestinal extracellular matrix, placental extracellular matrix, ornomentum extracellular matrix, cardiac extracellular matrix, kidney extracellular matrix, pancreas extracellular matrix, lung extracellular matrix, and combinations thereof.
